(19) United States
(12) Patent Application Publication
(10) Pub. No.: US 2023/0348832 A1
(43) Pub. Date: Nov. 2, 2023

(54) Cell Culture System

(71) Applicants: TERUMO KABUSHIKI KAISHA, Tokyo (JP); Terumo BCT, Inc., Lakewood, CO (US)

(72) Inventors: Masatsugu IGARASHI, Kanagawa (JP); Hirotaka OHASHI, Tokyo (JP)

(21) Appl. No.: 18/213,930

(22) Filed: Jun. 26, 2023

Related U.S. Application Data
(63) Continuation of application No. PCT/JP2022/012947, filed on Mar. 22, 2022.

(30) Foreign Application Priority Data
Mar. 26, 2021 (JP) 2021-052675

(51) Int. Cl. C12M 1/00; C12M 1/04; C12M 1/34

(52) U.S. Cl. CPC C12M 23/40; C12M 27/04; C12M 41/40; C12M 29/24

(57) ABSTRACT

A cell culturing system includes a reactor, a flow channel that allows a culture medium to flow into and out of the reactor, a waste liquid channel that discharges the culture medium from the flow channel, and a waste liquid container capable of storing the culture medium passing through the waste liquid channel. The waste liquid channel includes a temporary storage unit capable of temporarily storing the culture medium. The waste liquid container is located below the temporary storage unit) in a gravity direction, and the temporary storage unit is provided above the flow channel in the gravity direction. The temporary storage unit temporarily stores the culture medium discharged from the flow channel and allows the culture medium to flow out toward the waste liquid container.

### CROSS-REFERENCE TO RELATED APPLICATIONS

The present application is a continuation application of the International Patent Application No. PCT/JP2022/012947 filed on Mar. 22, 2022, which designated the U.S. and claims the benefit of priority from Japanese Patent Application No. JP2021-052675 filed on Mar. 26, 2021. The entire disclosures of the above-identified applications are incorporated herein by reference.

### FIELD

The present disclosure relates to a cell culturing system that cultures cells in a reactor by moving a culture medium into and out of the reactor.

### BACKGROUND

In regenerative medicine, cells of a living body are collected and cultured, and the cultured cells are administered to a patient. In cell culture treatment, for example, a cell culturing system may use a cell culturing container (also referred to as a reactor) that includes a hollow fiber disposed, for example, in a case. Cells may be seeded in the hollow fiber of the reactor and a culture medium fed into the reactor via a flow channel to culture the cells. The culture medium flowing out of the reactor during culture may be discharged to a waste liquid collection container (also referred to as a waste liquid unit).

In this type of cell culturing system, a waste liquid unit formed of a medical bag and the like may be hung on a stand and arranged above the reactor in a gravity direction. As a result, the cell culturing system may apply a positive pressure to the reactor and the flow channel via the culture medium flowing into the waste liquid unit and may suppress an excessive inflow of air (air bubbles) into the reactor.

However, in a case where a large amount of culture medium is discharged to the waste liquid unit over a long period of time and a small-capacity medical bag is used, replacement work of the waste liquid unit may be frequently required, increasing a workload of an operator. Even if the waste liquid unit includes a large tank, the operator still needs to remove the waste liquid unit that stores the larger amount of culture medium so that a load also increases.

Accordingly, there is a need for a cell culturing system capable of appropriately applying a positive pressure to a reactor and a flow channel and also capable of reducing a workload, including, for example, replacement and removal of a waste liquid unit.

### SUMMARY

In at least one example embodiment, the present disclosure provides a cell culturing system that includes a reactor that is configured to culture cells on the basis of a flow of a culture medium, a flow channel that allows the culture medium to flow into and out of the reactor, a waste liquid channel connected to the flow channel and configured to discharge the culture medium from the flow channel, and a waste liquid collection container connected to the waste liquid channel and capable of storing the culture medium passing through the waste liquid channel. The waste liquid channel may include a temporary storage unit that is capable of temporarily storing the culture medium. The waste liquid container may be positioned below the temporary storage unit in a gravity direction, and the temporary storage unit may be provided above the flow channel in the gravity direction. The temporary storage store may temporarily store the culture medium discharged from the flow channel and may allow the culture medium to flow out toward the waste liquid container.

In at least one example embodiment, the cell culturing system of the present disclosure may appropriately apply a positive pressure to a reactor and a flow channel, helping to reduce a workload in replacement, removal, and the like of the waste liquid unit.

### BRIEF DESCRIPTION OF DRAWINGS

FIG. 1 is a perspective view of a cell culturing system in accordance with at least one example embodiment of the present disclosure.

FIG. 2 is a circuit diagram illustrating a flow channel and a flow path control mechanism unit between a culture medium storage unit and a reactor for use with the cell culturing system illustrated in FIG. 1 in accordance with at least one example embodiment of the present disclosure.

FIG. 3 is a schematic illustrating an example waste liquid channel and an example waste liquid unit for use with the cell culturing system illustrated in FIG. 1 in accordance with at least one example embodiment of the present disclosure.

FIG. 4A is a partial perspective view illustrating an example temporary storage unit for use with the cell culturing system illustrated in FIG. 1 in accordance with at least one example embodiment of the present disclosure.

FIG. 4B is a cross-sectional view of the temporary storage unit illustrated in FIG. 4A.

FIG. 5 is a schematic illustrating another example waste liquid channel and another example waste liquid unit for use with the cell culturing system illustrated in FIG. 1 in accordance with at least one example embodiment of the present disclosure.

### DETAILED DESCRIPTION

Example embodiments of the present disclosure are hereafter described in detail with reference to the accompanying drawings.

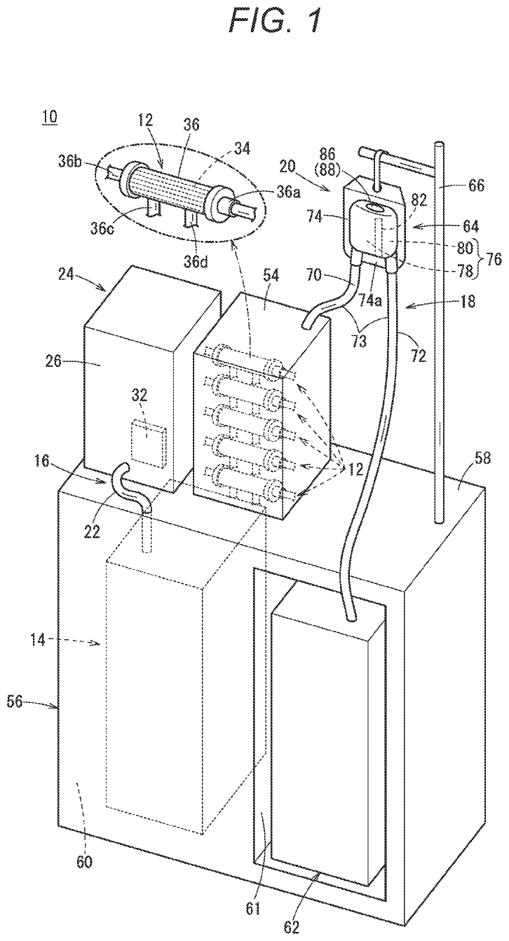

As illustrated in FIG. 1, a cell culturing system 10 according to at least one example embodiment of the present disclosure may be formed as a stationary device installed, for example, in a sterile room and configured to perform culture treatment to culture cells of a living body in regenerative medicine. The cell culturing system 10 may be equipped with a reactor 12, which is a cell culturing container. The cell culturing system 10 may discharge lactic acid, carbon dioxide, and/or the like (including, for example, unused culture medium and/or oxygen) generated during cell culture from the reactor 12 while supplying the culture medium and/or oxygen to the reactor 12, thereby performing the cell culture over a long period of time.

The cells of the living body are not limited, and may include, for example, cells contained in the blood (e.g., T cells and/or the like) and/or stem cells (e.g., ES cells, iPS cells, mesenchymal stem cells, and/or the like). An appropriate culture medium may be selected according to the cells of the living body. In at least one example embodiment, the culture medium may be prepared by adding amino acids, vitamins, serum, and/or the like to a basic solution. The basic solution may include, for example, a balanced salt solution (BSS).

In addition to the reactor 12, the cell culturing system 10 may include a culture medium storage unit 14 that is configured to store a culture medium, a flow channel 16 provided between the reactor 12 and the culture medium storage unit 14, a waste liquid channel 18 that discharges the culture medium from the flow channel 16, and a waste liquid unit 20 that is configured to store the culture medium that flows through the waste liquid channel 18. The cell culturing system 10 may include a plurality of reactors 12 (including, for example, five reactors 12, as illustrated in FIG. 1), thereby improving efficiency of the culture treatment. That is, the cell culturing system 10 may be configured to obtain several times the number of cells as compared to systems including only one reactor 12 without significantly changing a culturing period by allowing the culture medium to flow through each of the plurality of reactors 12 and culturing the cells in each reactor 12.

The culture medium storage unit 14 is configured to supply the culture medium to each reactor. A hard or soft tank capable of storing a large amount of culture medium may be used a as the culture medium storage unit 14. In at least one example embodiment, the tank has a volume of about 5 L to about 30 L, so as to reduce a workload of frequently replacing the culture medium storage unit 14 during the culture treatment. In at least one example embodiment, a flexible medical bag and/or the like may be applied as the culture medium storage unit 14.

Although, in FIG. 1, only the tube 22 connected to the culture medium storage unit 14 is illustrated. It should be appreciated that the flow channel 16 may include a plurality of tubes 22. Each tube 22 may be connected to the culture medium storage unit 14 and one or more medical bags (not illustrated), individual or collectively, and may be connected to each reactor 12, individually or collectively. As a result, the cell culturing system 10 may be configured to supply and discharge the culture medium in the culture medium storage unit 14 and the liquid (e.g., cell solution, cleaning solution, stripping solution, and/or the like) in each medical bag to and from the reactor 12 via each tube 22.

The cell solution may include a liquid containing cells to be cultured seeded in the reactor 12. The cleaning solution may include a liquid used when priming the reactor 12 and the flow channel 16. The cleaning solution may include buffer solutions and/or a physiological saline. The buffer solution may include phosphate buffered salts (PBS) and/or tris-buffered saline (TBS). The stripping solution may include a liquid that is selected to strip the cells cultured by the culture treatment. The stripping solution, may include, for example, trypsin, EDTA solution, and/or the like.

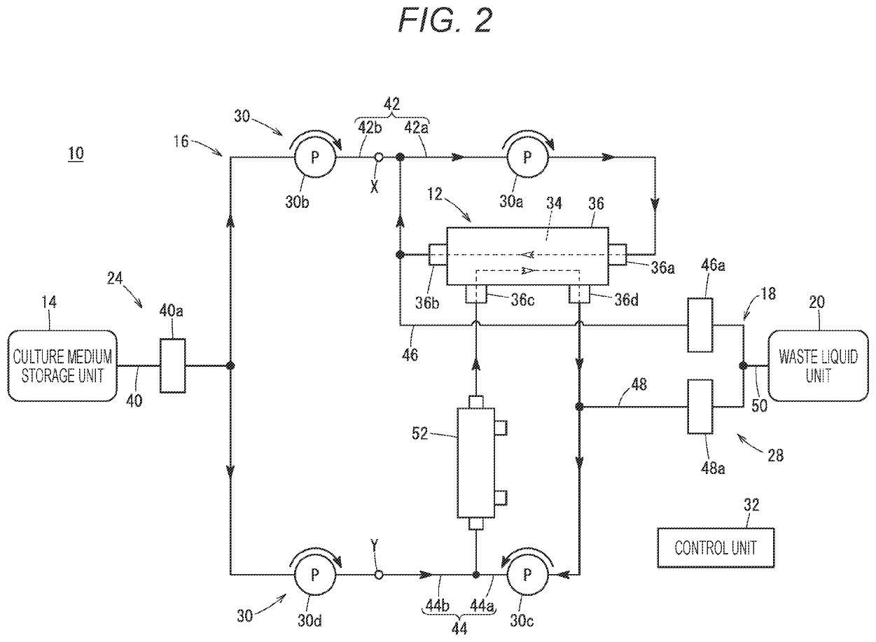

When the cell culturing system 10 is constructed, the flow channel 16 may be set so as to pass through a flow path control mechanism unit 24. The flow path control mechanism unit 24 may be equipped with a first casing 26 that accommodates a part of the flow channel 16. The flow path control mechanism unit 24 may be equipped with a plurality of clamps 28 that are configured to open and close a predetermined tube 22, a plurality of pumps 30 that are configured to allow the liquid in the tube 22 to flow, and/or a control unit 32 that is configured to control operation of each clamp 28 and/or each pump 30 in the first casing 26 (refer to FIG. 2). That is, the flow path control mechanism unit 24 may be configured to allow the liquid in the flow channel 16 to flow under the operation of the pump 30 while selectively switching the tube 22 through which the liquid flows by opening and closing the respective clamp 28.

In addition to the plurality of tubes 22, the flow channel 16 may include a cassette (not illustrated) that includes, for example, a plurality of liquid flow paths. The cassette may be connected to tube 22. The cassette may be set in the first casing 26, such that this opening/closing, switching, and the like of the flow path in the cassette may be performed by the clamp 28.

In order to increase a culturing area of each reactor 12 connected to the flow channel 16, a structure including a hollow fiber 34 may be used. Each reactor 12 may be equipped with a plurality of hollow fibers 34 (e.g., 10,000 or more) and a case 36 that accommodates the plurality of hollow fibers 34 in, for example, an axial direction.

Although not illustrated, it should be appreciated that each hollow fiber 34 may include an inner cavity penetrating in an extending direction and cells may be seeded on an inner peripheral surface forming the inner cavity. Each hollow fiber 34 may include a plurality of pores (not illustrated) through which the inner cavity communicates with the outside. Each pore may be configured so as not to permeate cells and/or proteins but to permeate a solution and/or a small-molecular substance. Therefore, the culture medium, a predetermined gas component, and/or the like may be supplied to the cells on the inner peripheral surface of the hollow fiber 34 through the pores. A configuration in which the liquid is allowed to flow principally through the inner cavity of the hollow fiber 34 may be referred to as intra capillary (IC), whereas a configuration in which the liquid is allowed to mainly flow outside the hollow fiber 34 may be referred to as extra capillary (EC).

The hollow fiber 34 may include polyolefin resins and/or polymer materials. The polyolefin resins may include as polypropylene and/or polyethylene. The polymer materials may include polysulfone, polyethersulfone, polyacrylonitrile, polytetrafluoroethylene, polystyrene, polymethylmethacrylate, cellulose acetate, cellulose triacetate, and/or regenerated cellulose.

The case 36 may have a cylindrical shape and may be rigid. The case 36 may include a first IC terminal 36a, a second IC terminal 36b, a first EC terminal 36c, and/or a second EC terminal 36d connected to each tube 22. The first IC terminal 36a may be provided at one end in the axial direction of the case 36 and may be configured to communicate with the inner cavity of the hollow fiber 34. The second IC terminal 36b may be provided at the other end in the axial direction of the case 36 and may be configured to communicate with the inner cavity of the hollow fiber 34. The first EC terminal 36c may be provided near the other end of a side surface of the case 36 and may be configured to communicate with a space outside the hollow fiber 34 in the case 36. The second EC terminal 36d may be provided near the one end of the side surface of the case 36 and may be configured to communicate with the space outside the hollow fiber 34 in the case 36.

Configurations of the flow channel 16 between one reactor 12 and the culture medium storage unit 14 and also the flow path control mechanism unit 24 are described with reference to FIG. 2.

The flow channel 16 may include a culture medium delivery route 40 connected to the culture medium storage unit 14 and an IC route 42 (internal route) and an EC route 44 (external route) branched from the culture medium delivery route 40. The IC route 42 may include a channel that supplies the liquid into the inner cavity of the hollow fiber 34 and the liquid (e.g., cleaning solution, cell solution, culture medium, and/or stripping solution) may flow therethrough. The EC route 44 may include a channel that supplies the liquid to the area between an outside of the hollow fiber 34 and an inside surface or wall of the case 36 and the liquid (e.g., the cleaning solution, culture medium, and/or stripping solution) may flow therethrough.

The culture medium delivery route 40 may include a first clamp 40a that is configured to permit or block the supply of the culture medium from the culture medium storage unit 14.

The IC route 42 may include an IC circulation circuit 42a capable of circulating the liquid with the reactor 12 and an IC supply circuit 42b capable of allowing the liquid to flow from the culture medium delivery route 40 to the IC circulation circuit 42a. The IC circulation circuit 42a may include an IC circulation pump 30a for circulating the liquid. The IC supply circuit 42b may include an IC supply pump 30b for allowing the liquid to flow from the culture medium delivery route 40 to the IC circulation circuit 42a. Although not illustrated, it should be appreciated that in addition to the culture medium storage unit 14, the tube 22 may be connected to one or more medical bags storing, for example, the cleaning solution, cell solution, stripping solution, and/or the like is connected to the IC supply circuit 42b.

The IC circulation circuit 42a may be connected to the first IC terminal 36a and the second IC terminal 36b of the reactor 12. Therefore, the liquid may circulate in the IC circulation circuit 42a and flow through the inner cavity of the hollow fiber 34 under the operation of the IC circulation pump 30a.

The IC waste liquid circuit 46 may be connected to a downstream side from the reactor 12 on the IC circulation circuit 42a. The IC waste liquid circuit 46 may form a part of the waste liquid channel 18 and may be connected to a merging route 50 of the waste liquid channel 18. The IC waste liquid circuit 46 may be provided with a second clamp 46a that is configured to permit or blocks discharge of the liquid from the IC circulation circuit 42a.

The EC route 44 may include an EC circulation circuit 44a capable of circulating the liquid with the reactor 12 and an EC supply circuit 44b capable of allowing the liquid to flow from the culture medium delivery route 40 to the EC circulation circuit 44a. The EC circulation circuit 44a may include an EC circulation pump 30c for circulating the liquid. The EC supply circuit 44b may include an EC supply pump 30d for allowing the liquid to flow from the culture medium delivery route 40 to the EC circulation circuit 44a. Although not illustrated, it should be appreciated that in addition to the culture medium storage unit 14, the tube 22 connected to one or more medical bags storing, for example, the cleaning solution, stripping solution, and/or the like may be connected to the EC supply circuit 44b.

The EC circulation circuit 44a may be connected to the first EC terminal 36c and the second EC terminal 36d of the reactor 12. Therefore, the liquid that circulates in the EC circulation circuit 44a may flow through the case 36 under the operation of the EC circulation pump 30c. A gas exchanger 52 may be provided on an upstream side from the reactor 12 on the EC circulation circuit 44a. The gas exchanger 52 may be configured to discharge carbon dioxide mixed in the culture medium and/or to mix a predetermined gas component (including, for example, nitrogen N₂: 75%, oxygen O₂: 20%, and/or carbon dioxide CO₂: 5%) with the culture medium. A structure of the gas exchanger 52 is not limited. It should be appreciated that a structure in which a plurality of hollow fibers is provided in a case may be used, as is the instance of the reactor 12.

An EC waste liquid circuit 48 may be connected to a downstream side from the reactor 12 on the EC circulation circuit 44a. The EC waste liquid circuit 48 may form a part of the waste liquid channel 18 and may be connected to the merging route 50 of the waste liquid channel 18. The EC waste liquid circuit 48 may be provided with a third clamp 48a that is configured to permit or blocks discharge of the liquid from the EC circulation circuit 44a.

In a case where a plurality of reactors 12 (e.g., five) is provided, the cell culturing system 10 may be equipped with a plurality of IC circulation circuits 42a and a plurality of EC circulation circuits 44a corresponding to the respective reactors 12. That is, another IC circulation circuit and another EC circulation circuit (not illustrated) that are configured to circulate the liquid in another reactor 12 may be connected in parallel to a branch point X between the IC supply pump 30b and the IC circulation circuit 42a and a branch point Y between the EC supply pump 30d and the EC circulation circuit 44a, respectively.

With renewed reference to FIG. 1, the cell culturing system 10 may include a second casing 54 that is configured to accommodate each reactor 12 at a position adjacent to the first casing 26 that forms, for example, the flow path control mechanism unit 24. The second casing 54 may be configured to keep a temperature of an accommodation chamber of each reactor 12 at about 37° C. That is, the cell culturing system 10 may easily form an environment suitable for the cell culture of each reactor 12 by using the second casing 54 different from the first casing 26 of the flow path control mechanism unit 24. It should be appreciated, however, that the cell culturing system 10 is not limited to the configuration in which each reactor 12 and the flow channel 16 are accommodated in a plurality of casings and may, in at least one example embodiment, be configured to accommodate them in one casing.

The second casing 54 may be formed to include a part (e.g., clamp 28, pump 30, and/or the like) of the flow path control mechanism unit 24 therein. For example, the second clamp 46a on the IC waste liquid circuit 46 and the third clamp 48a on the EC waste liquid circuit 48 may be provided in the second casing 54. The second casing 54 may be configured to rotatably fix each reactor 12 in a gravity direction or a horizontal direction and/or around the axis of the case 36. As a result, air in each reactor 12 may be easily discharged from the case 36.

The cell culturing system 10 may include an installation base 56 on which the first casing 26 and the second casing 54 may be installed. The installation base 56 may include a top plate 58 on which the first casing 26 and the second casing 54 are placed. The top plate 58 may be supported at a predetermined height (in at least one example embodiment ranging from about 50 cm to about 150 cm) by a side wall and/or the like of the installation base 56. The culture medium storage unit 14 may be accommodated in a culture medium accommodation box 60 of the installation base 56 as provided below the top plate 58.

The waste liquid channel 18 of the cell culturing system 10 may be connected to the flow channel 16 and may be connected to the waste liquid unit 20, thereby discharging the liquid (e.g., the culture medium, the cleaning solution, and/or the like) from the flow channel 16 to the waste liquid unit 20. The waste liquid channel 18 may include the IC waste liquid circuit 46 of the IC route 42, the EC waste liquid circuit 48 of the EC route 44, and/or the merging route 50 (refer to FIG. 2). The merging route 50 of the waste liquid channel 18 may be provided so as to be extended from the interior of the second casing 54 (or the first casing 26) to the outside.

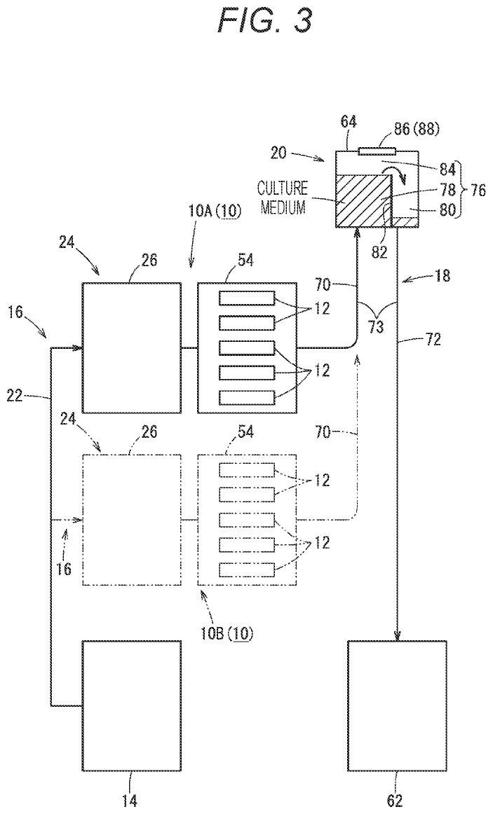

As illustrated in FIGS. 1 and 3, the waste liquid unit 20 may include a waste liquid container 62 that may be connected to the most downstream side of the waste liquid channel 18 and that is capable of storing the culture medium passing through the waste liquid channel 18. The waste liquid channel 18 may include a temporary storage unit 64 capable of temporarily storing the culture medium on an upstream side from the waste liquid container 62.

In the waste liquid container 62, a hard or soft tank having a large volume may be used for storing the culture medium used in each reactor 12. For example, the tank may have a volume ranging from about 5 L to about 30 L. As a result, a workload of frequently replacing the waste liquid container 62 may be reduced. Alternatively, a flexible medical bag and the like may be applied as the waste liquid container 62.

The waste liquid container 62 may be accommodated in a waste liquid accommodation box 61 of the installation base 56 as provided below the top plate 58. That is, the waste liquid container 62 may be provided below the temporary storage unit 64 in the gravity direction. In at least one example embodiment, the waste liquid container 62 may be located below the first casing 26 and the second casing 54 accommodating each reactor 12 in the gravity direction. Although FIG. 1 illustrates a state in which the waste liquid container 62 is exposed from the waste liquid accommodation box 61, it should be appreciated that, in at least one example embodiment, the waste liquid accommodation box 61 may have a configuration of hermetically sealing the waste liquid container 62.

The temporary storage unit 64 may be configured to temporarily stores the liquid discharged from the flow channel 16 and to allow the same to flow out toward the waste liquid container 62. Therefore, a volume of the temporary storage unit 64 may be sufficiently smaller than a volume of the waste liquid container 62. The temporary storage unit 64 may, for example, be hung on a stand 66 fixed to the installation base 56. The temporary storage unit 64 may be located above the first casing 26 and the second casing 54 accommodating the plurality of reactors 12 in the gravity direction. In other words, the temporary storage unit 64 may be arranged above the reactor 12 and the flow channel 16 in the gravity direction. A height of the temporary storage unit 64 is not especially limited. For example, in at least one example embodiment, the height of the temporary storage unit 64 may be in a range of from about 150 cm to about 180 cm. The height of the temporary storage unit 64 may be set to have a predetermined difference (for example, in a range of from about 10 cm to about 80 cm) with respect to the heights of the reactor 12 and the flow channel 16.

The waste liquid channel 18 (merging route 50) may include an upstream line 70 and a downstream line 72. The upstream line 70 may be provided between the flow channel 16 and the temporary storage unit 64 via the IC waste liquid circuit 46 and the EC waste liquid circuit 48. The downstream line 72 may be provided between the temporary storage unit 64 and the waste liquid container 62. The upstream line 70 and the downstream line 72 may be formed of a tube 73 including a flow path inside. The upstream line 70 may extended upward in the gravity direction from the second casing 54 and may be connected to a lower portion of the temporary storage unit 64. The downstream line 72 may extended downward in the gravity direction from the temporary storage unit 64 and may be connected to an upper portion of the waste liquid container 62.

In at least one example embodiment, a flexible medical bag may be used as the temporary storage unit 64. The temporary storage unit 64 may include a sealed portion 74 obtained by sealing outer peripheries of two sheets forming the medical bag. The temporary storage unit 64 may include a storage space 76 inside the sealed portion 74 and between the two sheets. The upstream line 70 and the downstream line 72 may be coupled to the sealed portion 74 on a lower side of the temporary storage unit 64 (referred to, for example, as a lower sealed portion 74a). The temporary storage unit 64 may include a hard container.

A partition wall 82 may be provided inside the temporary storage unit 64 to separate a lower side of the storage space 76 into a first storage unit 78 and a second storage unit 80. The partition wall 82 may continue to the lower sealed portion 74a and may extended upward in the gravity direction from the lower sealed portion 74a. The partition wall 82 may be formed by, for example, sealing the two sheets forming the medical bag. In another embodiment, the partition wall 82 may be formed by welding an edge of a plate member and each sheet in a state in which the plate member having a predetermined thickness in a direction orthogonal to two sheet surfaces may be interposed between the two sheets.

In the temporary storage unit 64, a communication unit 84 (a part of the storage space 76) through which the first storage unit 78 communicates with the second storage unit 80 may be provided above the partition wall 82 in the gravity direction. That is, the storage space 76 may include the communication unit 84 on the upper side in the gravity direction and the first storage unit 78 and the second storage unit 80 adjacent to each other in a lateral direction (direction orthogonal to the gravity direction) of the partition wall 82 below the communication unit 84 in the gravity direction. A flow path of the upstream line 70 fixed to the lower sealed portion 74a may communicate with the first storage unit 78. A flow path of the downstream line 72 fixed to the lower sealed portion 74a may communicate with the second storage unit 80.

Therefore, the liquid that flows into the temporary storage unit 64 from the upstream line 70 may be first stored in the first storage unit 78, and when the liquid fills the first storage unit 78, the liquid may go over the partition wall 82 and flow into the second storage unit 80. The liquid that flows into the second storage unit 80 may flow out to the downstream line 72 (outside the temporary storage unit 64).

The first storage unit 78 may be configured to apply an appropriate pressure (positive pressure) to the reactor 12 and the flow channel 16 via the stored culture medium. For example, a volume of the first storage unit 78 may be in a range of about 0.5 times to about three times a volume of the second storage unit 80. An actual volume of the first storage unit 78 may be set to, for example, a range of about 50 cc to about 300 cc.

The temporary storage unit 64 may include an atmosphere open unit 86 that applies an atmospheric pressure to the liquid that flows into the first storage unit 78. In at least one example embodiment, the atmosphere open unit 86 may include a vent mechanism 88 that permeates gas and blocks permeation of a liquid. As a result, the vent mechanism 88 may apply the atmospheric pressure to the liquid in the first storage unit 78 without leaking the liquid that flows into the storage space 76 to the outside. It should be appreciated, however, that the atmosphere open unit 86 is not limited to the vent mechanism 88 and in other embodiments may be formed of an opening that simply opens an upper side in the gravity direction of the temporary storage unit 64.

As illustrated in FIG. 3, the waste liquid unit 20 may be connected to a plurality of cell culturing systems 10. For example, by branching the upstream line 70 of the waste liquid channel 18, the flow channel 16 of a first cell culturing system 10A (solid line in FIG. 3) and the flow channel 16 of a second cell culturing system 10B (two-dot chain line in FIG. 3) may be connected to the temporary storage unit 64. Therefore, the waste liquid unit 20 may temporarily store both the liquid that flows out of the flow channel 16 of the first cell culturing system 10A and the liquid that flows out of the flow channel 16 of the second cell culturing system 10B in one temporary storage unit 64. Then, the liquid stored in the temporary storage unit 64 may be discharged to one or more waste liquid containers 62 via the downstream line 72.

As illustrated in FIG. 1, before the culture treatment is performed, an operator may set a plurality of reactors 12 in the second casing 54 of the cell culturing system 10 and may also set the flow channel 16 in the flow path control mechanism unit 24 of the cell culturing system 10. The operator may place the culture medium storage unit 14 in the culture medium accommodation box 60 of the installation base 56 and may install the waste liquid container 62 in the waste liquid accommodation box 61 of the installation base 56. The temporary storage unit 64 may be hung on the stand 66. As a result, the flow channel 16, for example, as illustrated in FIG. 2, may be constructed between the culture medium storage unit 14 and each reactor 12, and the temporary storage unit 64 may be arranged above the flow channel 16 in the gravity direction.

After the above-described setting, the cell culturing system 10 may be configured to perform a priming step, a culture medium replacement step, a seeding step, a culturing step, a stripping step, and/or a collection step in the culture treatment. During the priming step, the cleaning solution stored in a medical bag (not illustrated) may be supplied to each reactor 12 via the flow channel 16 and air may be removed from the reactor 12 and the flow channel 16. During the culture medium replacement step, the culture medium may be supplied from the culture medium storage unit 14 to each reactor 12 via the primed flow channel 16 and the inside and outside of the hollow fiber 34 may be filled with the culture medium. During the seeding step, the cell solution stored in a medical bag (not illustrated) may be supplied into the hollow fiber 34 of each reactor 12 via the IC route 42 and the cells may be seeded on the inner peripheral surface of the hollow fiber 34.

As illustrated in FIG. 2, at the culturing step, the cell culturing system 10 may be configured to supply the culture medium from the culture medium storage unit 14 to and into the hollow fiber 34 via both the IC route 42 and the EC route 44 and may culture the cells in the hollow fiber 34. At that time, carbon dioxide may be discharged from the culture medium and oxygen may be supplied to the culture medium by the gas exchanger 52. The culturing step may be performed for a longer period of time (for example, several days) than other steps, so that the cells may gradually propagate on the inner peripheral surface of the hollow fiber 34. The cell culturing system 10 may be configured to supply the culture medium to the reactor 12 via the EC route 44 without passing through the IC supply circuit 42b. The culture medium that flows through the EC route 44 to flow into the reactor 12 may move (oozes) from the outside to the inside of the hollow fiber 34 to be supplied to the cells.

During the culturing step, the culture medium that circulates in the IC circulation circuit 42a may flow to and into the IC waste liquid circuit 46 while the second clamp 46a is opened. The culture medium that circulates in the EC circulation circuit 44a may flow into the EC waste liquid circuit 48 while the third clamp 48a is opened. As a result, the culture medium may flow through the waste liquid channel 18. The culture medium in the IC waste liquid circuit 46 and the EC waste liquid circuit 48 may move to an outside the second casing 54 by flowing into the upstream line 70 of the merging route 50. This culture medium may flow upward in the gravity direction via the upstream line 70 and may flow into the first storage unit 78 of the temporary storage unit 64.

As illustrated in FIG. 3, the temporary storage unit 64 may be configured to continuously store the culture medium in the first storage unit 78 until the culture medium exceeds the partition wall 82. When the culture medium in the first storage unit 78 exceeds the partition wall 82, the culture medium may flow over the partition wall 82 (via the communication unit 84) and into the second storage unit 80. The culture medium moved to the second storage unit 80 may flow to the downstream line 72 fixed to a lower portion of the second storage unit 80. That is, when an inflow amount of the culture medium exceeds a certain amount, the temporary storage unit 64 may automatically discharge the culture medium to the downstream line 72 below the same.

The culture medium discharged to the downstream line 72 may flow downward in the gravity direction and may flow into the waste liquid container 62 installed on the lower side of the installation base 56. The waste liquid container 62 may have a volume capable of sufficiently storing the culture medium and may reduce the number of times of replacement of the waste liquid container 62 at the culturing step.

The temporary storage unit 64 arranged above the flow channel 16 and the reactor 12 in the gravity direction may apply the positive pressure to the flow channel 16 via the culture medium in the first storage unit 78 and the upstream line 70. Therefore, in the EC circulation circuit 44a, the positive pressure of the culture medium may be applied, so that it is possible to suppress an excessive inflow of air in the gas exchanger 52 and to stably mix the air and the culture medium. As a result, in the cell culturing system 10, the inflow of air bubbles into the flow channel 16 may be suppressed.

The atmosphere open unit 86 provided in the temporary storage unit 64 may be configured to apply the atmospheric pressure to the culture medium in the first storage unit 78 and the upstream line 70, thereby applying the positive pressure to the flow channel 16 even when the amount of the culture medium is small. Since the vent mechanism 88 may be employed as the atmosphere open unit 86, it may be possible to avoid leakage of the culture medium from the temporary storage unit 64.

During the stripping step after the culturing step, the cell culturing system 10 may be configured to guide the stripping solution stored in a medical bag (not illustrated) into the hollow fiber 34 of the reactor 12 via the IC route 42. The stripping solution may be selected to strip the cultured (propagated) cells. During the collection step, after the stripping step, the cell culturing system 10 may be configured to supply the culture medium to the IC route 42 to allow the cells stripped during the stripping step to flow out of the reactor 12 and to move to a collection bag (not illustrated).

Through the above-described steps, the cell culturing system 10 may satisfactorily store the cells cultured in the reactor 12 in the collection bag. For example, the cell culturing system 10 may stably apply the positive pressure to the flow channel 16 and may reduce a workload of the operator by reducing the number of times of replacement, removal, and/or the like of the waste liquid container 62 even when a large amount of culture medium is used.

It should be appreciated that the present disclosure is not limited to the above-described embodiments and various modifications may be made in accordance with different aspects of the disclosure. For example, in at least one example embodiment, the cell culturing system 10 may be configured to perform the culture treatment by one reactor 12 without using a plurality of reactors 12. In a case of increasing the number of cultured cells in the culture treatment, a larger sized reactor 12 may be applied.

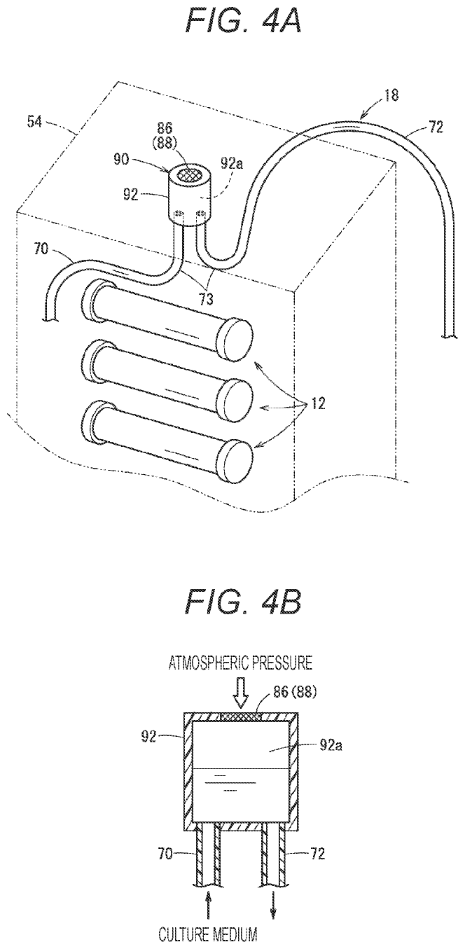

As illustrated in FIGS. 4A and 4B, in at least one example embodiment, the temporary storage unit 90 may be provided on an upper side of the second casing 54 that accommodates a plurality of reactors 12 (or the first casing 26, as illustrated in FIG. 1). As illustrated in FIGS. 4A and 4B, the temporary storage unit 90 may be arranged above the reactor 12 and the flow channel 16 in the gravity direction. For example, the temporary storage unit 90 may include a small and hard container 92 in such a manner that a volume does not change in the second casing 54 (other mechanisms are not pressed by the flexible bag).

The container 92 does not include a partition wall 82 (refer to FIG. 1). An inner surface of the container 92 may define a storage space 92a. Each of the upstream line 70 and the downstream line 72 of the waste liquid channel 18 may be connected to a lower end of the container 92 of the temporary storage unit 90. The upstream line 70 may extended upward in the gravity direction from a connection site of the flow channel 16 (refer to FIG. 2). In contrast, the downstream line 72 may be temporarily directed upward in the gravity direction from a lower portion of the container 92 in the second casing 54, thereby being exposed outside the second casing 54. Outside the second casing 54, the downstream line 72 may be extended downward in the gravity direction and may be connected to the waste liquid container 62 (refer to FIG. 1) installed below the second casing 54 in the gravity direction.

The atmosphere open unit 86 that applies the atmospheric pressure to the culture medium that flows into the storage space 92a may be provided on an upper end of the container 92 of the temporary storage unit 90. The atmosphere open unit 86 may include the vent mechanism 88 that permeates gas and blocks permeation of a liquid. The atmosphere open unit 86 may have a configuration in which the upper end of the container 92 is simply opened.

The temporary storage unit 90 as described above may also obtain an effect similar to that of the temporary storage unit 64 described above. That is, the culture medium that flows into the temporary storage unit 90 from the flow channel 16 via the upstream line 70 may be temporarily stored in the storage space 92a of the container 92. When the atmospheric pressure is applied to the culture medium in the container 92 from the atmosphere open unit 86, the positive pressure may be applied to the flow channel 16 via the culture medium of the upstream line 70. For example, the temporary storage unit 90 provided in the second casing 54 may avoid disadvantages such as dropping of the temporary storage unit 90 by an operator and/or the like.

The culture medium in the storage space 92a may be guided to the downstream line 72 by a siphon effect. The culture medium may be temporarily directed upward in the gravity direction in the downstream line 72, then guided downward in the gravity direction, and stored in the waste liquid container 62. As a result, the culture medium may be temporarily stored in the temporary storage unit 90 and then smoothly discharged to the waste liquid container 62. The culture medium does not accumulate in a large amount in the container 92.

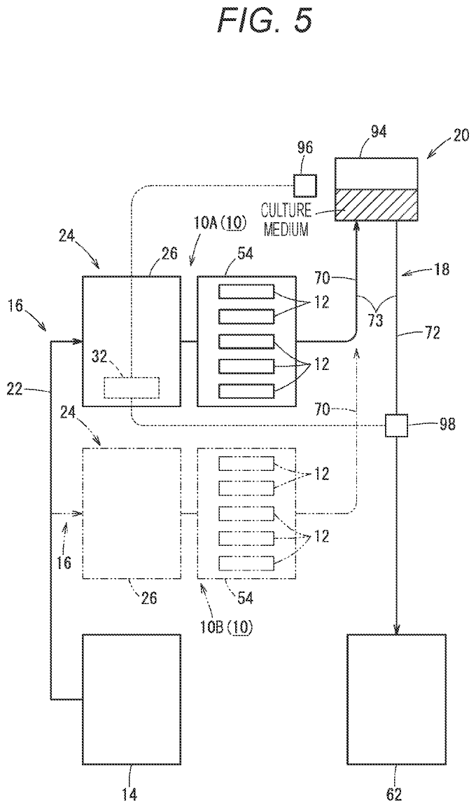

As illustrated in FIG. 5, in at least one example embodiment, the cell culturing system 10 may apply a temporary storage unit 94 without the partition wall 82 and may include a sensor 96 that is configured to detect a weight or a liquid level of the temporary storage unit 94 and a valve 98 that is configured to open and close the downstream line 72. The sensor 96 and the valve 98 may be connected to the control unit 32 of the cell culturing system 10 so as to be able to communicate by wire or wirelessly. The control unit 32 may be configured to cause the valve 98 to close in a normal state, to monitor the amount of liquid flowing into the temporary storage unit 94 on the basis of detection information of the sensor 96, and to cause the valve 98 to open in a case where the amount of liquid exceeds a predetermined threshold. The cell culturing system 10 may stably apply the positive pressure to the flow channel 16.

In at least one example embodiment, the present disclosure provides a cell culturing system 10 that includes a reactor 12 that may be configured to culture cells on the basis of a flow of a culture medium, a flow channel 16 that may be configured to allow the culture medium to flow into and out of the reactor 12, a waste liquid channel 18 that may be connected to the flow channel 16 and configured to discharge the culture medium from the flow channel 16, and a waste liquid container 62 that may be connected to the waste liquid channel 18 and that is configured to store the culture medium passing through the waste liquid channel 18. The waste liquid channel 18 may include a temporary storage unit 64, 90, 94 that is capable of temporarily storing the culture medium. The waste liquid container 62 may be located below the temporary storage unit 64, 90, 94 in a gravity direction, and the temporary storage unit 64, 90, 94 may be provided above the flow channel 16 in the gravity direction. The temporary storage unit 64, 90, 94 may be configured to temporarily store the culture medium discharged from the flow channel 16 and to allow the culture medium to flow out toward the waste liquid container 62.

In the cell culturing system 10, the temporary storage unit 64, 90, 94 may be located above the flow channel 16 in the gravity direction, so that the positive pressure may be appropriately applied to the reactor 12 and the flow channel 16 via the culture medium of the temporary storage unit 64, 90, 94. As a result, the cell culturing system 10 may suppress the excessive inflow of air into the reactor 12 and the flow channel 16. Since the waste liquid container 62 is located below the temporary storage unit 64, 90, 94 in the gravity direction, the cell culturing system 10 may reduce a workload in replacement, removal, and/or the like of the waste liquid container 62.

The waste liquid container 62 may be provided below an installation site of the reactor 12 in the gravity direction. This makes it easier for the operator of the cell culturing system 10 to perform operations, including, for example, replacement and/or removal of the waste liquid container 62.

A first storage unit 78, a second storage unit 80, a partition wall 82 that partitions the first storage unit 78 and the second storage unit 80, and a communication unit 84 through which the first storage unit 78 communicates with the second storage unit 80 above the partition wall 82 in the gravity direction may be provided inside the temporary storage unit 64, 94. The waste liquid channel 18 may include an upstream line 70 through which the flow channel 16 communicates with the first storage unit 78 and a downstream line 72 through which the second storage unit 80 communicates with the waste liquid container 62. The downstream line 72 may extended downward in the gravity direction from the second storage unit 80. As a result, the culture medium stored in the first storage unit 78 may apply an appropriate positive pressure to the reactor 12 and the flow channel 16.

The first storage unit 78 and the second storage unit 80 may be located at positions adjacent to each other in a direction orthogonal to the gravity direction. A volume of the first storage unit 78 may be larger than a volume of the second storage unit 80. As a result, the temporary storage unit 64 may apply a large positive pressure from the first storage unit 78 to the reactor 12 and the flow channel 16.

The temporary storage unit 90 may include an atmosphere open unit 86 that applies an atmospheric pressure to the culture medium that flows into the temporary storage unit 90. The atmosphere open unit 86 may include a vent mechanism 88 that permeates gas and blocks permeation of a liquid. As a result, even in the configuration in which the cell culturing system 10 includes the atmosphere open unit 86, leakage of the culture medium from the temporary storage unit 90 may be suppressed.

The waste liquid channel 18 may include an upstream line 70 through which the flow channel 16 communicates with the temporary storage unit 90 and a downstream line 72 through which the temporary storage unit 90 communicates with the waste liquid container 62. The downstream line 72 may be temporarily directed upward in the gravity direction from a lower portion of the temporary storage unit 90 and then directed downward in the gravity direction. In such instances, the cell culturing system 10 may discharge the culture medium from the temporary storage unit 90 to the waste liquid container 62 while applying the positive pressure to the reactor 12 and the flow channel 16 by the culture medium stored in the temporary storage unit 90.

The temporary storage unit 64, 94 may be arranged outside a casing (second casing 54) that accommodates the reactor 12. As a result, the operator may easily set the temporary storage unit 64, 94 when preparing the cell culturing system 10. The operator may visually check the culture medium in the temporary storage unit 64, 94 located outside and recognize a waste liquid state of the culture medium.

The temporary storage unit 90 may be arranged inside a casing (second casing 54) that accommodates the reactor 12. As a result, the cell culturing system 10 may avoid disadvantages, including, for example, dropping of the temporary storage unit 90 by the operator or the like.

The flow channel 16 may include a circulation circuit (EC circulation circuit 44a) that circulates the culture medium with the reactor 12 and a supply circuit (EC supply circuit 44b) that supplies the culture medium to the circulation circuit. The circulation circuit may include a gas exchanger 52 that mixes gas with the culture medium on an upstream side from the reactor 12 in a flow direction of the culture medium and the waste liquid channel 18 that may be connected to a downstream side from the reactor 12 in the flow direction of the culture medium. As a result, the cell culturing system 10 may apply an appropriate positive pressure to the circulation circuit including the gas exchanger 52 via the culture medium in the temporary storage unit 64, 90, 94 and the waste liquid channel 18 and may suppress an excessive inflow of gas in the gas exchanger 52.

A plurality of reactors 12 may be provided, and the culture medium that flows through the plurality of reactors 12 may be collectively discharged to the waste liquid channel 18 and the waste liquid container 62. The cell culturing system 10 may efficiently culture the cells by the plurality of reactors 12. The cell culturing system 10 may stably waste the culture medium by the waste liquid container 62 and the temporary storage unit 64, 90, 94 by using the plurality of reactors 12 even if a large amount of the culture medium flows.

1. A cell culturing system comprising:
a temporary storage unit configured to temporarily store a culture medium used for cell culturing; and
a waste liquid container in fluid communication with the temporary storage unit and located below the temporary storage unit in a gravity direction.

2. The cell culturing system of claim 1, wherein the cell culturing system further includes:
a reactor that is configured to culture cells, the reactor in fluid communication with the temporary storage unit and the culture medium flowing from the reactor to the temporary storage unit.

3. The cell culturing system of claim 2, wherein the temporary storage unit is arranged outside a casing that accommodates the reactor.

4. The cell culturing system of claim 2, wherein the temporary storage unit is arranged inside a casing that accommodates the reactor.

5. The cell culturing system of claim 2, further comprising a flow channel configured to allow the culture medium to flow into and out of the reactor.

6. The cell culturing system of claim 5, wherein the flow channel includes:
a circulation circuit that provides the culture medium to the reactor; and
a supply circuit that provides the culture medium to the circulation circuit.

7. The cell culturing system of claim 6, wherein the circulation circuit includes a gas exchanger that is configured to mix gas with the culture medium at an upstream side of the reactor and in a flow direction of the culture medium.

8. The cell culturing system of claim 5, wherein the waste liquid container is provided below an installation site of the reactor in the gravity direction.

9. The cell culturing system of claim 5, wherein the cell culturing system further includes:
a waste liquid channel that connects to the flow channel and that is configured to discharge the culture medium from the flow channel.

10. The cell culturing system of claim 9, wherein the waste liquid channel includes an upstream line through which the flow channel communicates with the temporary storage unit and a downstream line through which the temporary storage unit communicates with the waste liquid container.

11. The cell culturing system of claim 10, wherein the downstream line is moveable between an upward position in the gravity direction and a downward position in the gravity direction.

12. The cell culturing system of claim 9, wherein the temporary storage unit is provided above the flow channel in the gravity direction.

13. The cell culturing system of claim 9, wherein the temporary storage unit includes a first storage unit, a second storage unit, a partition wall that separates the first storage unit and the second storage unit, and a communication unit located above the partition wall in the gravity direction and through which the first storage unit communicates with the second storage unit.

14. The cell culturing system of claim 13, wherein the waste liquid channel includes an upstream line through which the flow channel communicates with the first storage unit and a downstream line through which the second storage unit communicates with the waste liquid container.

15. The cell culturing system of claim 14, wherein the downstream line is extended downward in the gravity direction from the second storage unit.

16. The cell culturing system of claim 12, wherein the first storage unit and the second storage unit are positioned adjacent to each other in a direction orthogonal to the gravity direction. and

17. The cell culturing system of claim 16, wherein a volume of the first storage unit is larger than a volume of the second storage unit.

18. The cell culturing system of claim 1, wherein the temporary storage unit includes an atmosphere open unit that is configured to apply an atmospheric pressure to the culture medium, the atmosphere open unit including a vent mechanism that permeates gas and blocks permeation of a liquid.

19. A cell culturing system comprising:
a reactor that is configured to culture cells using a culture medium;
a temporary storage unit configured to temporarily store the culture medium used for cell culturing, the temporary storage unit including a first storage unit, a second storage unit, a partition wall that separates the first storage unit and the second storage unit, and a communication unit located above the partition wall in a gravity direction and through which the first storage unit communicates with the second storage unit; and
a waste liquid container in fluid communication with the temporary storage unit and located below the temporary storage unit in the gravity direction.

20. The cell culturing system of claim 19, wherein the temporary storage unit includes an atmosphere open unit that is configured to apply an atmospheric pressure to the culture medium, the atmosphere open unit including a vent mechanism that permeates gas and blocks permeation of a liquid.
